(19) United States
(12) Patent Application Publication
(10) Pub. No.: US 2012/0245070 A1
(43) Pub. Date: Sep. 27, 2012

(54) Use Of Branched Alkyl(Oligo)Glycosides In Cleaning Agents

(75) Inventors: Rainer Eskuchen, Langenfeld (DE); Sabine Both, Neuss (DE); Arnold Benert, Neuss (DE); Ditmar Kischkel, Monheim (DE)

(73) Assignee: Cognis IP Management GmbH, Dusseldorf (DE)

(21) Appl. No.: 13/513,762

(22) PCT Filed: Nov. 26, 2010
(86) PCT No.: PCT/EP2010/007177

§ 371 Date: Jun. 4, 2012

(30) Foreign Application Priority Data
Dec. 5, 2009 (EP) EP09015094

(51) Int. Cl. C11D 3/22; C07H 3/04; C07H 3/06; C07H 3/02

(52) U.S. Cl. 510/218; 510/470; 510/238; 536/1.11; 536/123.13; 536/123.1

(57) ABSTRACT

Disclosed is the use of alkyl(oligo)glycosides based on selected branched fatty alcohols as hydrotropic agents, preferably for use in cleaning agents, in particular agents for automatic dish washing.

The present invention relates to the use of certain branched alkyl (oligo)glycosides in cleaning agents.

Alkyl (oligo)glycosides are nonionic surfactants that have been known for a long time which are used both in cosmetics, but also in washing and cleaning agents. The advantages of these surfactant classes are in particular their good biodegradability, but also the fact that these surfactants can be obtained from renewable raw materials. Alkyl and alkenyl oligoglycosides conform to the formula R′O-[G]ₚ in which R′ is an alkyl and/or alkenyl radical having 4 to 22 carbon atoms, G is a sugar radical having 5 or 6 carbon atoms and p is numbers from 1 to 10. They can be obtained by the relevant methods of preparative organic chemistry. By way of representation of the extensive literature, reference may be made here to the review work by Biermann et al. in Starch 45, 281(1993), and also J. Kahre et al. in SÖFW-Journal volume 8, 598 (1995). The alkyl and/or alkenyl oligoglycosides can be derived from aldoses or ketoses having 5 or 6 carbon atoms, preferably glucose. The preferred alkyl and/or alkenyl oligoglycosides are thus alkyl and/or alkenyl oligoglucosides. The index number p in the general formula (I) indicates the degree of oligomerization (DP), i.e. the distribution of monoglycosides and oligoglycosides, and is a number between 1 and 10. Whereas p in a given compound must always be a whole number and here in particular can assume the values p=1 to 6, the value p for a specific alkyl (oligo)glycoside is an analytically determined calculated parameter, which in most cases is a fraction.

Preference is given to using alkyl and/or alkenyl (oligo)glycosides with an average degree of oligomerization p of 1.1 to 3.0.

As well as alkyl (oligo)glycosides based on unbranched fatty alcohols, those compounds which contain branched radicals R′ are also known. USH 171 already discloses alkyl (oligo)glycosides where the alcohol moiety in the 2 position can be branched, and e.g. an ethyl or propyl radical is disclosed as branching. The specification also discloses the suitability of this type of branched alkyl (oligo)glycosides for washing and cleaning agents. EP 0 690 868 A1 specifically discloses cleaning agents where the agents comprise branched alkyl (oligo)glycosides. The branched alcohols specified are in particular Guerbet alcohols, and namely 2-ethylhexyl alcohol and 2-propylheptyl alcohol. EP 1 292 660 A1 describes single-phase microemulsions which comprise branched alkyl (oligo) glycosides.

However, it has now surprisingly been found that these branched alkyl (oligo)glycosides have special properties which make them interesting for a large number of applications.

A first subject matter of the present application therefore relates to the use of alkyl (oligo)glycosides according to the general formula (I)

R—O-[G]ₓ (I)

in which G is a glycoside radical, x is a number from 1 to 3 and R is a 2-propylheptyl radical, as hydrotope for aqueous systems.

The compounds of formula (I) are known. Their preparation is described e.g. in USH 171.

In this connection, the reaction with the glycoses takes place quite generally in the presence of 2-propylheptanol (with the empirical formula C₁₀H₂₂O; CAS No. 10042-59-8).

This substance is also referred to as 2-propylheptan-1-ol or unsystematically called “propylheptanol”. The alkyl (oligo)glycosides of the present teaching have—depending on the reaction conditions—a degree of oligomerization, also called DP (in formula (I), the index x), in the range from 1 to and preferably from 1.0 to 2.0. Hexoses and here in particular glucose are selected as preferred sugar radical. However, pentoses are also possible as sugar radicals.

Hydrotropy is the term used to refer to the phenomenon where one sparingly soluble substance becomes water-soluble in the presence of a second component which is itself not a solvent. Substances which bring about a solubility improvement of this type are referred to as hydrotropes or hydrotropic agents. They act as solubility promoters with different action mechanisms: substances which have a tendency to form association colloids, such as e.g. surfactants, are able to permit, as a result of the formation of micelles, e.g. the solubility of long-chain alcohols that are otherwise insoluble in water. Typical hydrotropes, which are used e.g. in the formulating of liquid washing agents, are xylene or cumenesulfonate. Other substances, e.g. urea or N-methylacetamide, increase the solubility by virtue of a structure-breaking effect in which the water structure is broken down in the vicinity of the hydrophobic group of a sparingly soluble substance. An increase in solubility can also be effected as a result of the formation of mixed crystals with the hydrotropic substance in the sediment of the component to be dissolved. Solutions of hydrotropic substances (hydrotropic solutions) are used instead of organic solvents for extraction purposes. Advantage: the solutions are nonvolatile, nonflammable and nontoxic, can be regenerated easily and generally have higher HLB values. Particular preference is currently given to cumenesulfonate, although this is not biodegradable.

For the purposes of the present teaching, alkyl (oligo)glycosides of the formula (I) are selected in which R is a 2-propylheptyl radical.

The use of alkyl (oligo)glycosides of the formula (I) takes place preferably in amounts of from 0.5 to 10% by weight, preferably 1 to 5% by weight and in particular in amounts of from 2 to 4% by weight, based on the aqueous system. Suitable aqueous systems are both washing and cleaning agents, but also cosmetic preparations. For the purposes of the present technical teaching, aqueous systems comprise at least 50% by weight of water and preferably 50 to 99% by weight, or in particular 65 to 95% by weight of water. Preferably, the alkyl (oligo)glycosides according to the formula (I) are used as hydrotropes in cleaning agents and here preferably in aqueous all-purpose cleaners, dishwashing agents and bath cleaners.

The use in dishwashing agents is particularly preferred since the alkyl (oligo)glycosides of the formula (I) have good compatibility with plastics and here in particular the tendency towards so-called crack corrosion is lower than with other nonionic surfactants. Moreover, they exhibit an improved cleaning performance compared with linear alkyl (oligo)glycosides or alkyl (oligo)glycosides based on 2-ethylhexanol.

The branched alkyl (oligo)glycosides of the present invention used according to the invention exhibit comparable properties to cumenesulfonate, but are more biodegradable and are based on natural, renewable raw materials.

A further subject matter of the present application relates to dishwashing agents which comprise alkyl (oligo)glycosides according to the above formula (I), with the proviso that no further alkyl (oligo)glycosides with branched alkyl radicals or branched unsaturated alkenyl radicals are present in the agents.

The dishwashing agents can be solid or gel-like, but preferably liquid, and comprise further ingredients known per se, such as solvents (e.g. water or alcohols such as ethanol, propanol, isopropanol and butanol), surfactants, bleaches, enzymes, organic or inorganic acids, polymers, dyes, perfume or corrosion protectants or additives for preventing the tarnishing of silver or for the protection of glass or plastic. The alkyl (oligo)glycosides according to the invention as per formula (I) are preferably present in the agents in amounts of from 0.5 to 10% by weight.

Preferably, the agents comprise only compounds of the general formula (I) as the sole constituent of the alkyl (oligo)glycoside type. In this connection, alkyl (oligo)glycosides are understood as meaning those compounds of the formula R′—O-[G]_{y}, in which R′ is a linear or branched alkyl radical or an unsaturated, optionally branched alkenyl radical having 1 to 22 carbon atoms, G is a glycoside radical and y is numbers from 1 to 8, preferably from 1.0 to 3.0.

### EXAMPLES

### 1. General Preparation Procedure for the Synthesis of the 2-propylheptyl glycosides

A mixture of the glucose used and the majority of the 2-propylheptyl alcohol was placed in a 4 l stirred reactor with stirrer, reflux condenser, water separator, distillate receiver, vacuostat and vacuum pump. After reaching the reaction temperature, the catalyst dissolved in some of the alcohol used was metered into the reaction mixture over the course of 0.5 h. The water produced in the course of the reaction was distilled off continuously and collected in the distillate receiver via the water separator. The reaction was ended as soon as water of reaction was no longer produced. The acidic catalyst in the reaction mixture was neutralized with magnesium oxide and 50% strength NaOH solution. The content of excess alcohol was separated off in a manner known per se at elevated temperature and reduced pressure (180° C., <1 mbar). The propylheptyl glucoside was then diluted with water to give a paste with a solids concentration of 65-75%. This aqueous solution was bleached at 90° C. to a Hazen color number <50 with the addition of hydrogen peroxide (35% strength) and sodium hydroxide solution (20% strength). The Hazen color number was measured using the Lico 300 colorimeter from Dr. Lange. For the measurement, the glucoside solution is diluted to 10% active substance and filtered through a 0.45μ filter into a 11 mm round cuvette. The following three products according to the invention were prepared in accordance with the general procedure, as was a 2-ethylhexyl glycoside for comparison:

### Example 1

| | |
|---|---|
| 1812.60 g | 2-propylheptanol (11.45 mol) |
| 629.80 g | glucose monohydrate (3.18 mol) |
| 3.81 g | sulfosuccinic acid 70% strength (0.013 mol) |
| 0.71 g | magnesium oxide |
| 1.78 g | sodium hydroxide solution (50% strength) |

Reaction temperature: 110° C.
Pressure: 30 mbar
Degree of DP: 1.39
Active substance content of the aqueous paste: 65.4% by weight

### Example 2

| | |
|---|---|
| 2036.60 g | 2-propylheptanol (12.87 mol) |
| 536.10 g | xylose (3.57 mol) |
| 8.60 g | dodecylbenzenesulfonic acid (0.0275 mol) |
| 0.71 g | magnesium oxide |
| 1.84 g | sodium hydroxide solution (50% strength) |

Reaction temperature: 102° C.
Pressure: 30 mbar
Degree of DP: 1.24
Active substance content of the aqueous paste: % by weight 66.4%

### Example 3

| | |
|---|---|
| 1421.8 g | 2-propylheptanol (8.98 mol) |
| 889.2 g | glucose monohydrate (4.49 mol) |
| 7.70 g | dodecylbenzenesulfonic acid (0.0246 mol) |
| 0.71 g | magnesium oxide |
| 2.20 g | sodium hydroxide solution (50% strength) |

Reaction temperature: 110° C.
Pressure: 30 mbar
Degree of DP: 1.81
Active substance content of the aqueous paste: % by weight 58.7%

### Example 4

### Comparison

| | |
|---|---|
| 1172.1 g | 2-ethylhexanol (9 mol) |
| 540.5 g | glucose anhydrous (3 mol) |
| 7.70 g | dodecylbenzenesulfonic acid (0.0177 mol) |
| 0.28 g | magnesium oxide |
| 1.13 g | sodium hydroxide solution (50% strength) |

Reaction temperature: 100° C.
Pressure: 40 mbar
Degree of DP: 1.51
Active substance content of the aqueous paste: 49.4% by weight

### 2 Application Investigations

### 2.1 Foaming Behavior

A foaming measurement apparatus was used to test and compare the intrinsic foaming behavior of the propylheptyl glucosides and three other alkyl polyglucosides.

The foaming apparatus permits a dynamic “Ross-Miles” test. Here, the surfactant liquid is circulated continuously and pumped at the liquid level. In this method, the intrinsic foaming behavior of active substances or formulations is tested as a function of the temperature. In the case of solid samples, a presolution should always be prepared, unless the dissolution behavior is being investigated. 500 ml of distilled water are poured into the jacketed 2-liter measuring cylinder of the free-fall circulation apparatus. In the thermostat, the desired temperature profile (8° C. to 80° C. in 45 minutes) is opened and heated to the corresponding starting temperature ±1° C. in the measuring cylinder.

As soon as the starting temperature is reached, the temperature profile and also the hose pump are started. 0.2 ml of the substance or formulation to be tested are pipetted at the same time as 0.2 ml of active substance into the circulated water. Moreover, the stopwatch should be started. The resulting total volume (foam and liquid) is recorded with the associated temperatures and times. The measurement values in areas with a more rapid change in foam height are recorded at shorter intervals in order to depict the progression more precisely.

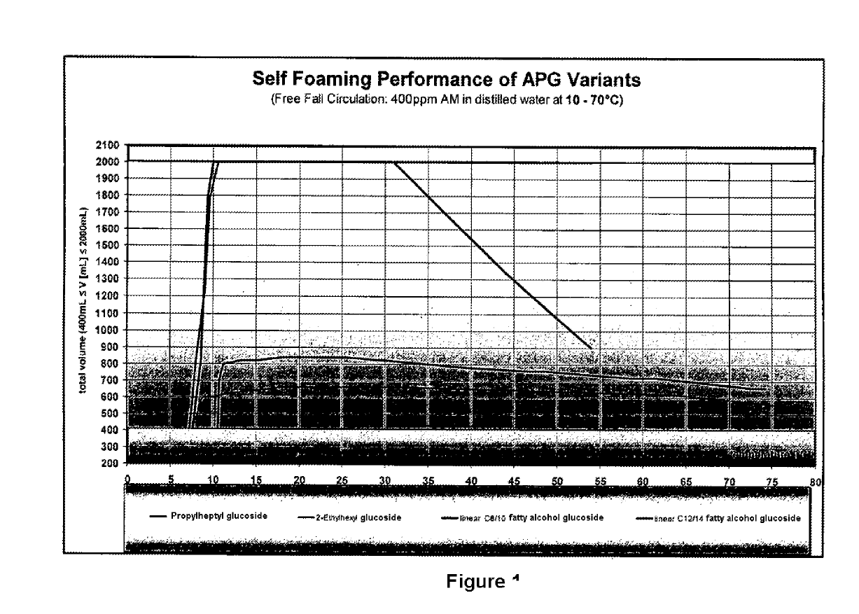

The result of the temperature-dependent foam height has been shown for 4 alkyl polyglucosides in FIG. 1. Here, a C8/10 fatty alcohol glucoside (Glucopon 225 DK), a C12/14 fatty alcohol glucoside (Glucopon 600 UP), a 2-ethylhexyl glucoside (Berol AG 6202, Akzo Nobel) and the APG from example 1 were tested.

It is clearly evident in FIG. 1 that the APGs constructed with linear fatty alcohol are highly foaming surfactants in the measurement arrangement since the maximum foam height of 200 ml are reached. The longer C12/14 chain length of the fatty alcohol foams over the entire temperature range, the short C8/10 linear chain becomes slowly lower-foaming above 35° C. Only at ca. 50° C. would the foam be at an acceptable level for an industrial cleaning application.

The branched 2-ethylhexyl APG exhibits a low foam height over the entire temperature range. The APG based on propylheptanol originating from example 1 shows a similar foaming behavior over the entire temperature range. The low-foaming capacity is comparable with the 2-ethylhexyl APG.

### Foam Quality:

Product examples 2, 3 and 4 were investigated as to whether they can improve the properties of surfactant formulations with regard to the aforementioned requirements.

### Test Formulation:

9%* Texapon NSO
3%* Dehyton PK 45
2%* substance to be tested
ad 100 dist. water

Procedure: Texapon NSO, Dehyton PK 45 and the substance to be tested are weighed in in succession, topped up to 100 g with dist. water and mixed. If necessary, citric acid is used to adjust the pH. A 2.5% strength solution is prepared from this formulation using hard water (15° German hardness). This is then beaten using a Mizer disk for 10 s at 2000 revolutions in an 800 ml beaker. For this, the stirrer is operated for 10 s at 2000 revolutions. The foam is spooned off onto a Ceran plate using a spatula spoon and photographed. To evaluate the foam, the height of the foam in the beaker is measured and the foam quality is evaluated using the benchmarks listed below with grades from 1-6, with 1 being the best foam quality and 6 being the worst foam quality. Table 3 gives the foam heights for 4 different APGs:

TABLE 3

| 9%* Texapon NSO + 3% Dehyton PK45 | Foam height | Foam quality |
|---|---|---|
| + — | 6.5 cm | 4-5 |
| + 2%* Propylheptyl xyloside (Ex. 2) | 10.0 cm | 1-2 |
| + 2%* Propylheptyl glucoside (Ex. 3) | 9.5 cm | 1-2 |
| + 2%* Ethylhexyl glucoside (Ex. 4) | 6.0 cm | 2-3 |
| + 2%* Plantacare 1200 UP | 5.5 cm | 3-4 |

*based on active substance (AS)

As the examples in table 3 show, the foam height and the foam structure of surfactant-containing formulations is considerably improved by adding propylheptyl glycosides. The examples with the ethylhexyl glucoside and the Plantacare 1200 UP used as comparison show that other alkyl polyglycosides exhibit these properties to a considerably lesser extent.

### 2.2 Determination of the Cleaning Performance of the Alkyl Polyglucosides Investigated:

The cleaning performance of the investigated APGs was carried out by means of a modified and automated Gardner test. The essential features of the test method were published as IPP quality standard in SÖFW 112. 10/1986 (Gardner test).

The method is based on the fact that a white soiling carrier treated with test soiling is wiped, under defined conditions, with a sponge saturated with the test material. The cleaning effect is measured by means of digital image evaluation against the untreated soiling carrier. For the purposes of better comparability, all glucosides were neutralized so that in the test only the cleaning performance of the surfactant and not in the presence of alkali is measured. Table 4 shows the cleaning performance of the 4 investigated APGs:

| | Measured |
|---|---|
| | cleaning |
| | performance |
| Component at 1% by weight (AS) | in % |
| Linear C8/10 fatty alcohol glucoside, neutralized | 68 |
| Linear C12/14 fatty alcohol glucoside, neutralized | 61 |
| Linear C8 fatty alcohol glucoside, neutralized | 49 |
| 2-Ethylhexyl glucoside, neutralized | 55 |
| Glucoside from example 1, propylheptyl glucoside, | 90 |
| neutralized | |

It is clearly evident that the linear fatty alcohol glucosides do have a good cleaning performance. The propylheptyl glucoside according to the invention exhibits a significantly better cleaning performance than the comparison products.

### 2.3. Hydrotropy Measurements

In industrial cleaning processes, alkyl polyglucosides are used as solubility promoters, especially in alkaline applications, in order to formulate foam-controlling surfactants into cleaning formulations, i.e. to obtain a homogeneous cleaning solution. However, the foam-controlling surfactants are generally difficult to formulate into aqueous systems on account of their hydrophobicity. Table 5 shows how much hydrotrope (=solubility promoter) has to be added in order to obtain the introduced components in a clear and homogeneous solution.

Mixture 1: 75% by weight water, 10% by weight NaOH, 10% NTA (nitrilotriacetic acid), 5% by weight Dehypon LS 54

Mixture 2: 75% by weight water, 10% by weight NaOH, 15% by weight Dehypon LS 36

TABLE 5

| | Addition in % by | Addition in % by |
|---|---|---|
| | weight (AS) to | weight (AS) to |
| Components | mixture 1 | mixture 2 |
| Sodium cumenesulfonate | 10 | 15 |
| C8/10 FA glucoside | 25, then solution | 35 |
| | still cloudy | |
| 2-Ethylhexyl glucoside | 15 | 25 |
| Propylheptyl glucoside | 16 | 18 |

(AS) = active substance

In the alkaline formulation with a large amount of hydrophobic surfactant, the propylheptyl glucoside exhibits significantly better solubility promoter properties than the linear fatty alcohol glucoside and the 2-ethylhexyl glucoside.

### 2.4. Plastic Compatibility

Here, the stress-crack corrosion of the materials is investigated. Stress-crack corrosion test on plastic strips in accordance with DIN 53449 T 1-3. The method was described in SÖFW 130 10-2004 pp. 83-93.

A stainless steel pin is pressed vertically into the plastic test strips, provided with a bore, using an apparatus. The sample strips are then briefly immersed into the medium to be tested. Adhering solution is not removed. The immersion process is repeated every 24 hours and carried out a total of 5 times. The test strip is evaluated every 24 hours.

Evaluation

| | | | 0-7 d | 0-14 d |
|---|---|---|---|---|
| 1 | No attack | → | recommended | recommended |
| 2 | slight cracks | → | of limited suitability | suitable |
| 3 | crack right through | → | of limited suitability | of limited suitability |
| 4 | broken through | → | of limited suitability | of limited suitability |

In the test, the particularly critical substrates were chosen. Table 6 shows the results:

TABLE 6

| | PMMA | | PC | | ABS | |
|---|---|---|---|---|---|---|
| | Plexiglas 8 N | | Makrolon 3103 | | Terez 3010 | |
| | 7 d | 14 d | 7 d | 14 d | 7 d | 14 d |
| Propylheptyl glucoside | 1 | 1 | 1 | 1.5 | 1 | 1 |
| C8/10 fatty alcohol | 1 | 1 | 1 | 1 | 1 | 1 |
| glucoside, linear | | | | | | |
| Fatty alcohol ethoxylate | 3 | 4 | 3 | 3 | 3 | 3 |
| 2-Ethylhexyl glucoside | 1 | 1.5 | 2 | 2.75 | 2 | 2 |

It is evident here that the linear and the propylheptyl glucoside have the best material compatibilities and are therefore suitable for use on these surfaces. The fatty alcohol ethoxylate and the 2-ethylhexyl glucoside do not exhibit good material compatibility.

1.-4. (canceled)

5. A method for improving the hydrotropic characteristics of an aqueous cleaning composition comprising incorporating into the aqueous cleaning composition an alkyl (oligo) glycoside according to the general formula (I)
R—O-[G]ₓ (I)
in which G is a glycoside radical, x is a number from 1 to 3 and R is a 2-propylheptyl radical.

6. The method of claim 5, wherein the alkyl (oligo) glycoside of formula (I) promotes solubility of a foam controlling surfactant in the composition.

7. The method of claim 5, wherein the alkyl (oligo) glycoside of formula (I) is incorporated into the aqueous cleaning composition in an amount of about 0.5 to 10% by weight.

8. The method of claim 5, wherein the alkyl (oligo) glycoside of formula (I) is incorporated into the aqueous cleaning composition in an amount of about 1 to 5% by weight.

9. The method of claim 5, wherein the alkyl (oligo) glycoside of formula (I) is incorporated into the aqueous cleaning composition in an amount of about 2 to 4% by weight.

10. The method of claim 5, wherein the alkyl (oligo) glycoside of formula (I) is incorporated into an aqueous washing agent, all-purpose cleaner, dishwashing agent, bath cleaner, or cosmetic agent.

11. The method of claim 5, wherein the aqueous cleaning composition has improved compatibility with plastic.

12. The method of claim 5, wherein the alkyl (oligo) glycoside of formula (I) is incorporated into an aqueous cleaning composition which comprises a foam controlling surfactant.

13. The method of claim 5, wherein the aqueous cleaning composition has improved foam height or foam quality.

14. A cleaning agent comprising an alkyl (oligo) glycoside of formula (I),
R—O-[G]ₓ (I)
wherein G is a glycoside radical, x is a number from 1 to 3 and R is a 2-propylheptyl radical; and
wherein the cleaning agent is substantially free of other alkyl (oligo) glycosides.

15. The cleaning agent of claim 14, which comprises 0.5 to 10% by weight of the alkyl (oligo) glycoside of formula (I).

16. The cleaning agent of claim 15, which comprises 1 to 5% by weight of the alkyl (oligo) glycoside of formula (I).

17. The cleaning agent of claim 16, which comprises 2 to 4% by weight of the alkyl (oligo) glycoside of formula (I).

18. The cleaning agent of claim 14, wherein the glycoside radical contains 5 or 6 carbons.

19. The cleaning agent of claim 14, wherein the glycoside radical of the alkyl (oligo) glycosides of formula (I) is glucoside or xyloside.

20. The cleaning agent of claim 14, further comprising a foam controlling surfactant.

21. The cleaning agent of claim 14, which exhibits improved compatibility with plastic.

22. The cleaning agent of claim 14, which exhibits improved foam height or foam quality.
